(19) United States
(12) Patent Application Publication
(10) Pub. No.: US 2023/0363850 A1
(43) Pub. Date: Nov. 16, 2023

(54) HEAD STABILIZATION SIMULATION SYSTEM

(71) Applicant: pro med instruments GmbH, Freiburg Im Breisgau (DE)

(72) Inventors: Jan H. Mertens, Freiburg (DE); Matthias E. Schuele, Freiburg (DE)

(21) Appl. No.: 18/317,491

(22) Filed: May 15, 2023

Related U.S. Application Data
(60) Provisional application No. 63/342,309, filed on May 16, 2022.

(51) Int. Cl. A61B 90/14; G09B 23/30; A61B 90/00

(52) U.S. Cl. CPC A61B 90/14; A61B 90/36; G09B 23/30; A61B 2090/365

(57) ABSTRACT

A head stabilization system useable for simulating stabilizing a head of a patient during a medical procedure includes a skull clamp with an adapter assembly that selectively receives a computing device configured to execute an application. Within a virtual reality environment, the skull clamp is represented by a virtual skull clamp and the head of the patient is represented by a virtual head of a virtual patient. Movements of the skull clamp based on instructions or feedback from the application, or from user’s unguided use appear on a display as movements of the virtual skull clamp relative to the virtual head of the virtual patient. The application can also guide the user in manipulating the skull clamp to achieve a successful stabilization. The system is usable for education and training.

### PRIORITY

This application claims priority to U.S. Provisional Pat. Application Serial No. 63/342,309, filed May 16, 2022, entitled “Head Stabilization Simulation System,” the disclosure of which is incorporated by reference herein.

### BACKGROUND

During certain medical procedures it may be necessary or desirable to stabilize all or a portion of a patient such that the patient or portion of the patient is immobilized. In certain neurological procedures the portion stabilized may include the head and/or neck of the patient. Certain devices and methods may be used to stabilize a certain portion of the patient. For example, a skull clamp is a type of head stabilization device that may be used to stabilize the head and/or neck of the patient. Skull clamps are typically manually adjusted relative to a skull of the patient to apply a sufficient amount of force onto the skull to stabilize the patient. If the skull clamp is not correctly positioned relative to the skull, slippage can occur during the medical procedure that can cause problems. Accordingly, it is desirable to have sound education and training of those users that may be charged with applying a head stabilization device such as a skull clamp to the head of a patient during certain medical procedures. While a variety of head stabilization devices and method of use of the same have been made and used, it is believed that no one prior to the inventor(s) has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements.

FIG. 1 depicts a front perspective view of an exemplary skull clamp for use in simulating a patient stabilization.

FIG. 2 depicts a rear perspective view of the skull clamp of FIG. 1.

FIG. 3 depicts a front perspective view of another exemplary skull clamp for use in simulating a patient stabilization.

FIG. 4 depicts a rear perspective view of the skull clamp of FIG. 3.

FIG. 5 depicts a front view of another exemplary skull clamp for use in simulating a patient stabilization.

FIG. 6 depicts a rear view of the skull clamp of FIG. 5.

FIG. 7 depicts a schematic view of an exemplary computing device, application, and connected devices usable with the skull clamps described herein.

FIGS. 8 and 9 depict flowcharts of an exemplary method of using the application and systems described herein to simulate stabilizing a patient’s head.

FIG. 10 depicts a schematic view of an exemplary physical skull clamp and an exemplary virtual skull clamp and virtual patient’s head shown on an exemplary display.

FIG. 11 depicts a view of a stabilized head of a patient along with reference markings.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

### I. Exemplary Skull Clamp With Adapter Assembly

As mentioned above, properly applying a skull clamp to a patient to stabilize them for a medical procedure is an important aspect of the overall medical procedure. Accordingly, having the ability to simulate a patient stabilization using a head fixation or stabilization device such as a skull clamp can be beneficial in term of training users as well as planning strategies and techniques to be used during an actual procedure. The following sections and paragraphs describe exemplary simulation systems.

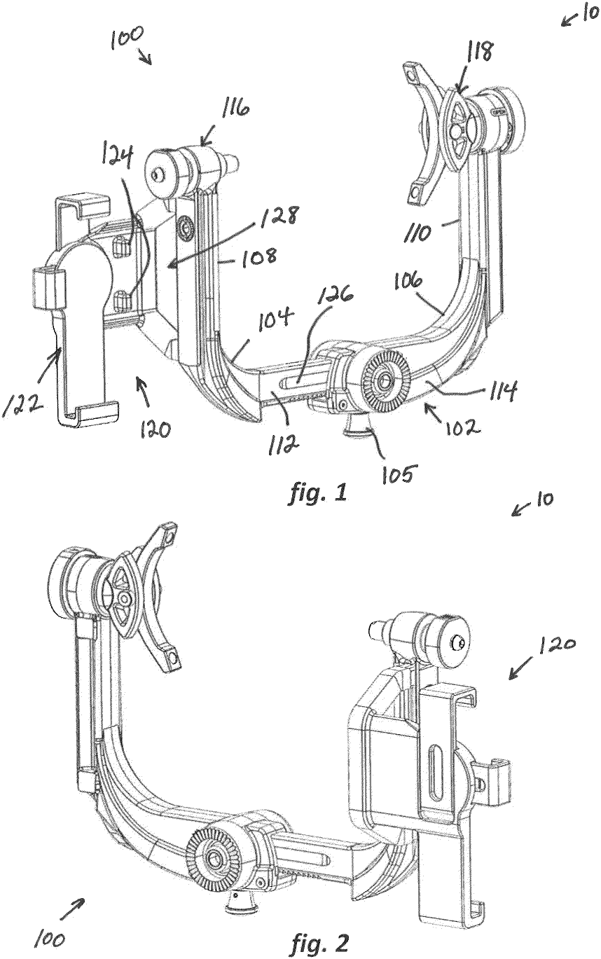

FIGS. 1-4 illustrate an exemplary head stabilization simulation system 10 that includes a head fixation or stabilization device in the form of a skull clamp 100. Skull clamp 100 includes a frame 102 made up of a first arm 104 and a second arm 106. Each arm 104, 106 defines a respective upright portion 108,110 and a lateral or base portion 112, 114. First and second arms 104, 106 are adjustable in their relative position to change a space between them to accommodate patient heads of various size. For instance, in the present example, skull clamp 100 includes a ratchet system that allows first arm 104 and second arm 106 to be moved closer together. This ratchet system of skull clamp 100 further includes an actuator 105 that can be used to release or disengage the ratchet system so that first arm 104 and second arm 106 can be moved further apart from one another.

First arm 104 includes a stabilization assembly 116, and second arm 106 includes a stabilization assembly 118. Each stabilization assembly 116, 118 is configured to receive at least one stabilization feature (not shown), which may be a skull pin in some examples, or a pad in some other examples. Furthermore, skull clamp 100 may be configured to use a combination of stabilization feature types, i.e., some pins and some pads in the same use configuration. As will be appreciated by those of ordinary skill in the art, skull clamp 100 is selectively connectable with other structures, such as one or more of a swivel adapter, a base unit, an operating table, etc. when used to stabilize the head of a patient.

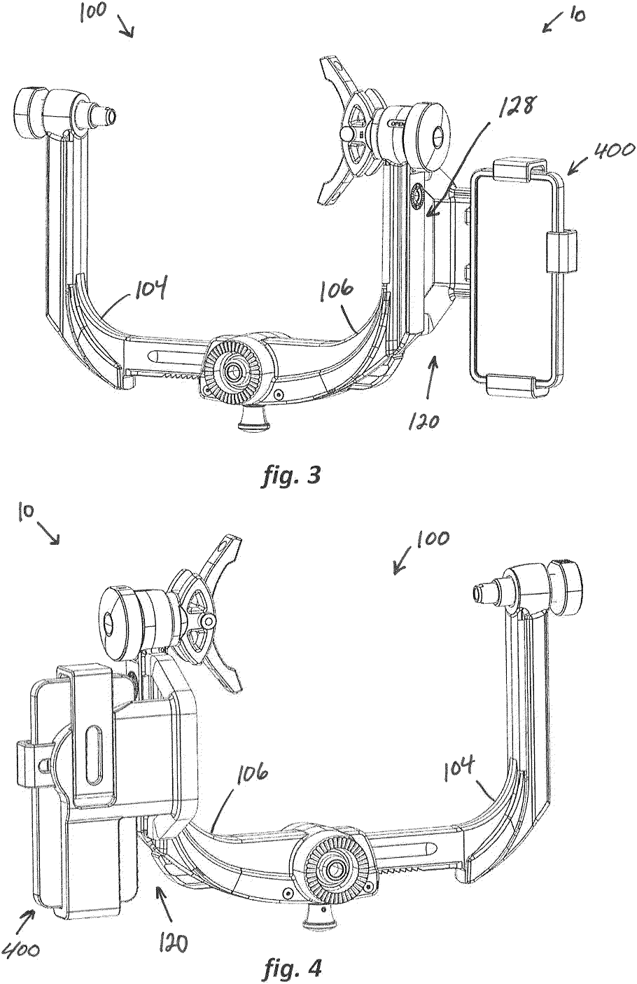

Skull clamp 100 also includes an adapter assembly 120 that is configured to selectively receive a computing device, such as computing device 400 discussed later. Adapter assembly 120 is connectable with skull clamp 100, and this connection can be either a fixed or permanent connection or a selective connection whereby adapter assembly 120 may be selectively connectable with skull clamp 100. In the fixed or permanent connection setup, adapter assembly 120 can be formed in unison or unitarily with skull clamp 100. In the present example shown in FIGS. 1 and 2, adapter assembly 120 is formed as part of first arm 104 of skull clamp 100. In the present example shown in FIGS. 3 and 4, adapter assembly 120 is formed as part of second arm 106 of skull clamp 100. Still in other versions, those of ordinary skill in the art will appreciate that adapter assembly 120 may be detachable and re-attachable with skull clamp 100 at either first arm 104 or second arm 106. Irrespective of the connection means with skull clamp 100, adapter assembly 120 is connectable with skull clamp 100 in a consistent and repeatable way such that the position (i.e., coordinates in three-dimensional system), and orientation (i.e., tilt or angulation about axes in three-dimensional system) of adapter assembly 120 relative to skull clamp 100 is known or defined.

In the present example, adapter assembly 120 includes a cradle 122 that is configured to hold or receive the computing device. Cradle 122 is configured to be able to selectively retain the computing device in a consistent position relative to skull clamp 100. In this manner, the computing device is held by cradle 122 the same way each time the computing device is connected with cradle 122. In the present example, to facilitate consistent mounting or connection of the computing device with cradle 122, there is a pair of fixed stops 124 positioned closest to skull clamp 100, where the mounted computing device would contact on one side as seen in, e.g., FIG. 3 with respect to skull clamp 100 and computing device 400. Based on the known position and orientation of adapter assembly 120, and the consistent and repeatable mounting of the computing device with cradle 122 of adapter assembly 120, a position and an orientation of the computing device relative to skull clamp 100 is known or defined. In this manner, the position and orientation of the computing device can be correlated to the position and orientation of skull clamp 100.

As seen best in FIGS. 1 and 3, adapter assembly 120 is shaped or configured to include a void space 128, which provides access for a user to grasp frame 102 of skull clamp 100. In this manner, a user is able to hold and manipulate or move skull clamp 100 as they usually would in a real procedure without interference from adapter assembly 120. More specially in the present example, a user is able to grasp skull clamp 100 about its first and second arms 104, 106 without interference from adapter assembly 120.

In one example, skull clamp 100 is fabricated by rapid prototyping or 3D printing, which may not be suitable for use in actual physical patient stabilizations. In such an example, adapter assembly 120 can be formed as part of the larger skull clamp 100. In some versions where skull clamp 100 is rapid prototype or a 3D printed structure, various weights 126 may be added to skull clamp 100 such that skull clamp 100 may have a weight and feel the same or similar to that of a skull clamp used in an actual patient stabilization procedure.

### II. Exemplary Skull Clamp With Dual Adapter Assembly

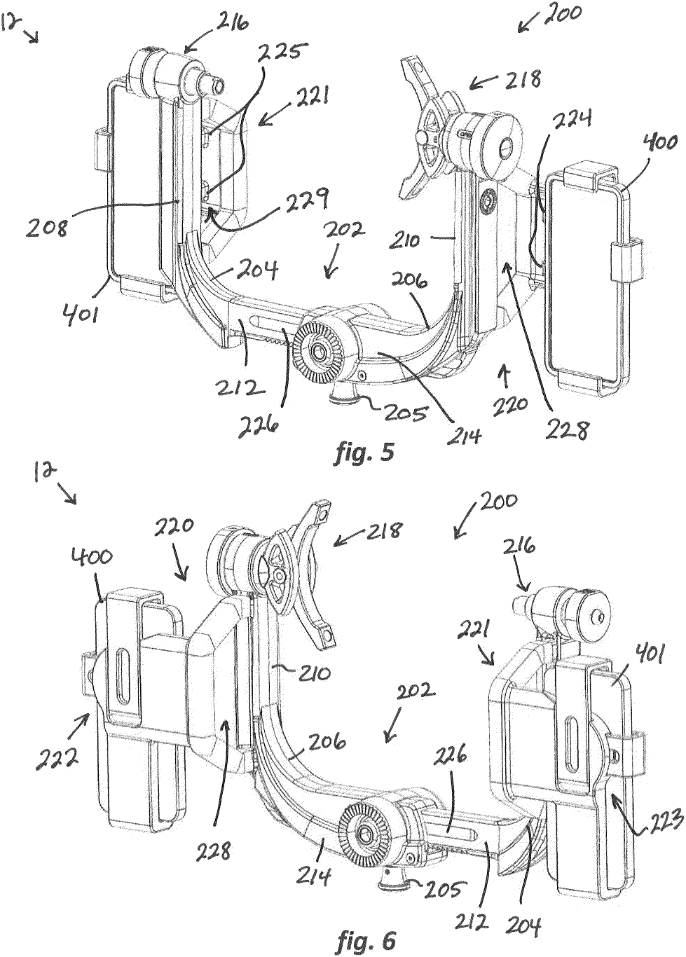

FIGS. 5 and 6 depict another exemplary head stabilization simulation system 12 that includes skull clamp 200, which comprises adapter assemblies 220, 221. In the present example, skull clamp 200 is identical to skull clamp 100 described above, except that instead of having only one adapter assembly, skull clamp 200 has a pair of adapter assemblies 220, 221. To this extent, skull clamp 200 includes a frame 202 made up of a first arm 204 and a second arm 206. Each arm 204, 206 defines a respective upright portion 208, 210 and a lateral or base portion 212, 214. First and second arms 204, 206 are adjustable in their relative position to change a space between them to accommodate patient heads of various size. For instance, in the present example, skull clamp 200 includes a ratchet system that allows first arm 204 and second arm 206 to be moved closer together. This ratchet system of skull clamp 200 further includes an actuator 205 that can be used to release or disengage the ratchet system so that first arm 204 and second arm 206 can be moved further apart from one another.

First arm 204 includes a stabilization assembly 216, and second arm 206 includes a stabilization assembly 218. Each stabilization assembly 216, 218 is configured to receive at least one stabilization feature (not shown), which may be a skull pin in some examples, or a pad in some other examples. Furthermore, skull clamp 200 may be configured to use a combination of stabilization feature types, i.e., some pins and some pads in the same use configuration. As will be appreciated by those of ordinary skill in the art, skull clamp 200 is selectively connectable with other structures, such as one or more of a swivel adapter, a base unit, an operating table, etc. when used to stabilize the head of a patient.

Regarding adapter assemblies 220, 221, each is configured to selectively receive a computing device, such as computing devices 400, 401 discussed later. Adapter assembly 220 is connectable with second arm 206 of skull clamp 200, while adapter assembly 221 is connectable with first arm 204 of skull clamp 200. Furthermore, the connection of adapter assemblies 220, 221 with skull clamp 200 can be either a fixed or permanent connection or a selective connection whereby either or both of adapter assemblies 220, 221 may be selectively connectable with skull clamp 200. In the fixed or permanent connection setup, adapter assemblies 220, 221 can be formed in unison or unitarily with skull clamp 200. Still in other versions, those of ordinary skill in the art will appreciate that adapter assemblies 220, 221 may be detachable and re-attachable with skull clamp 200 at respective first arm 204 and second arm 206. Irrespective of the connection means with skull clamp 200, adapter assemblies 220, 221 are connectable with skull clamp 200 in a consistent and repeatable way such that the position (i.e., coordinates in three-dimensional system), and orientation (i.e., tilt or angulation about axes in three-dimensional system) of each adapter assembly 220, 221 relative to skull clamp 200 is known or defined.

In the present example, each adapter assembly 220, 221 includes a respective cradle 222, 223 that is configured to hold or receive the computing device. In the present example, cradle 222 receives computing device 400, while cradle 223 receives computing device 401. Cradles 222, 223 are configured to be able to selectively retain the respective computing devices 400, 401 in a consistent position relative to skull clamp 200. In this manner, the computing devices 400, 401 are held by the respective cradles 222, 223 the same way each time the computing device 400, 401 is connected with the respective cradle 222, 223. In the present example, to facilitate consistent mounting or connection of computing devices 400, 401 with respective cradles 222, 223, each adapter assembly 220, 221 includes a pair of fixed stops 224, 225 positioned closest to skull clamp 200, where the respective mounted computing device 400, 401 would contact on one side as shown in FIG. 5. Based on the known position and orientation of each adapter assembly 220, 221, and the consistent and repeatable mounting of computing devices 400, 401 with respective adapter assemblies 220, 221, a position and an orientation of computing devices 400, 401 relative to skull clamp 200 is known or defined. In this manner, the position and orientation of computing devices 400, 401 can be correlated to the position and orientation of skull clamp 200.

With skull clamp 200 having dual adapter assemblies 220, 221 and two computing devices 400, 401, in some versions the position and orientation of computing device 401, e.g., is known based on communication between computing devices 400, 401. For instance, one computing device can be the primary computing device for the initial position and orientation and the other computing device can be the secondary computing device where its position and orientation is determined relative to the primary computing device.

As mentioned above, skull clamp 200 includes the ability for a width adjustment by changing the position of first and second arms 204, 206 relative to each other. A benefit of skull clamp 200 with dual adapter assemblies 220, 221 and computing devices 400, 401 is that the width setting or configuration of skull clamp 200 can be determined. For instance, not only can the position and orientation of skull clamp 200 be correlated to one or both of computing devices 400, 401, but based on communication between computing devices 400, 401 a distance between them can be determined and correlated to a distance between first and second arms 204, 206, thereby indicating the width setting or configuration of skull clamp 200.

As seen best in FIGS. 5 and 6, adapter assemblies 220, 221 are shaped or configured to each include a void space 228, 229, which provides access for a user to grasp frame 202 of skull clamp 200. In this manner, a user is able to hold and manipulate or move skull clamp 200 as they usually would in a real procedure without interference from adapter assemblies 220, 221. More specially in the present example, a user is able to grasp skull clamp 200 about its first and second arms 204, 206 without interference from adapter assemblies 220, 221.

Regarding fabrication, in one version skull clamp 200 is fabricated by rapid prototyping or 3D printing, which may not be suitable for use in actual physical patient stabilizations. In such an example, adapter assemblies 220, 221 can be formed as part of the larger skull clamp 200. In some versions where skull clamp 200 is a rapid prototype or 3D printed structure, various weights 226 may be added to skull clamp 200 such that skull clamp 200 may have a weight and feel the same or similar to that of a skull clamp used in an actual patient stabilization procedure.

### III. Exemplary Computing Device, Application, and Connected Devices

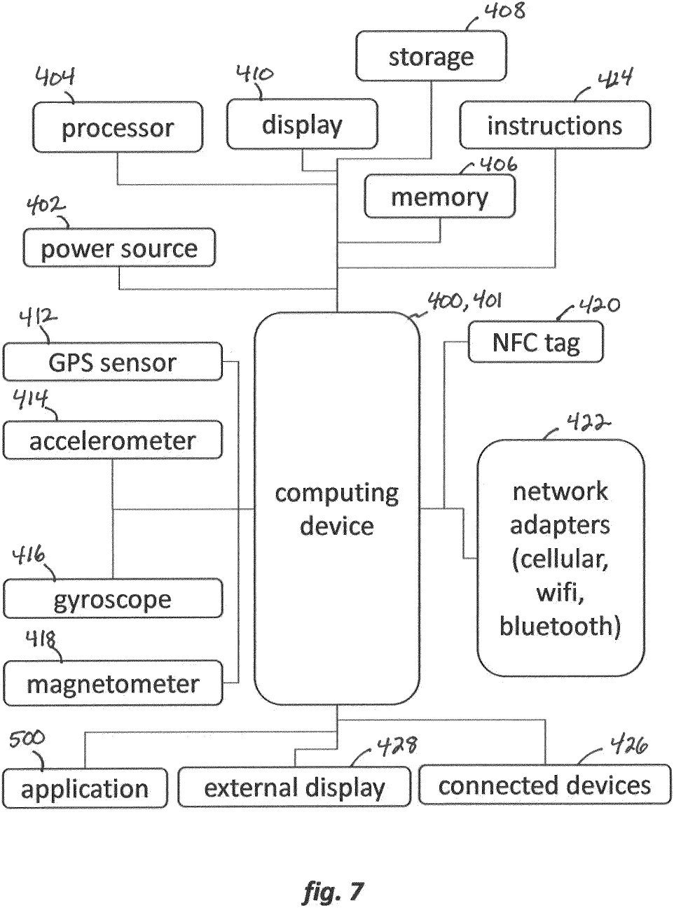

FIG. 7 shows a schematic view of an exemplary computing device 400. Computing device 401, in some versions, is configured the same as computing device 400, and therefore it should be understood that the following description of computing device 400 can apply equally to computing device 401.

As shown in FIG. 7, computing device 400 includes a power source or supply 402, which in some instances is a battery. Computing device 400 also includes a processor 404, a memory 406, a storage 408, and a display 410. In the present example, display 410 also serves as an input device as display 410 is configured as a touchscreen display. Computing device 400 also includes various sensors, such as a GPS sensor 412, an accelerometer 414, a gyroscope 416, and a magnetometer 418. Computing device 400 also includes a near field communication (NFC) tag 420 as well as network devices or adapters 422 that include among others, a cellular adapter, a bluetooth adapter, and a Wi-Fi adapter. Computing device 400 also includes one or more sets of instructions 424. In one instance one set of instructions 424 is saved locally on storage 408 and/or memory 406 and comprises an operating system software to operate computing device 400. In such version or other versions, another of the one or more sets of instructions 424 comprises application 500 as discussed further below.

Computing device 400 can be associated with connected devices 426 that may be external to computing device 400 and either wired or wirelessly connected with computing device 400. In one instance, an exemplary connected device 426 comprises an external display 428. In one example external display 428 is a monitor located remote from computing device 400. In another example connected devices 426 includes glasses or goggles equipped with a display such that a user can wear the glasses or goggles and view content received from computing device 400 and displayed on the glasses or goggles. Still in another example, connected devices 426 includes a separate computing device that may be detected and/or paired with computing device 400.

As shown in FIG. 7, an application 500 is associated with computing device 400. Application 500 comprises a set of instructions that are executable by computing device 400. In some versions, application 500 is a software application that can be installed locally on computing device 400 and run from computing device 400. In other versions, application 500 is a software application that may be only partially installed locally and accessed and run from a location remote from computing device 400. In this manner application 500 can be located in the cloud and part of a cloud computing architecture where all or a portion of application 500 is accessible via the internet. In this example, application 500 may be available on various mobile computing platforms, including Android and Apple, e.g., in their respective app stores. Once downloaded from an app store onto computing device 400, application 500 can be launched from computing device 500. Launching application 500 opens a simulation software configured to operate in conjunction with a head fixation or stabilization system such as skull clamps 100, 200 described above.

In view of the teachings herein, those of ordinary skill in the art will appreciate various ways of using computing device 400 in conjunction with connected devices 426 and application 500. Furthermore, those of ordinary skill in the art will appreciate various ways of creating computer executable code and/or instructions to carry out the steps and process of the stabilization simulation described herein.

### IV. Exemplary Use

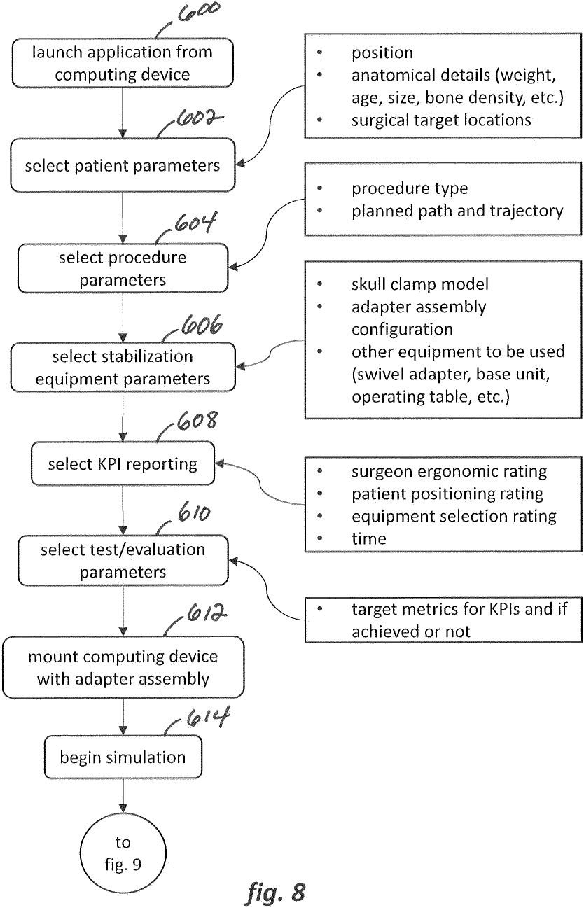

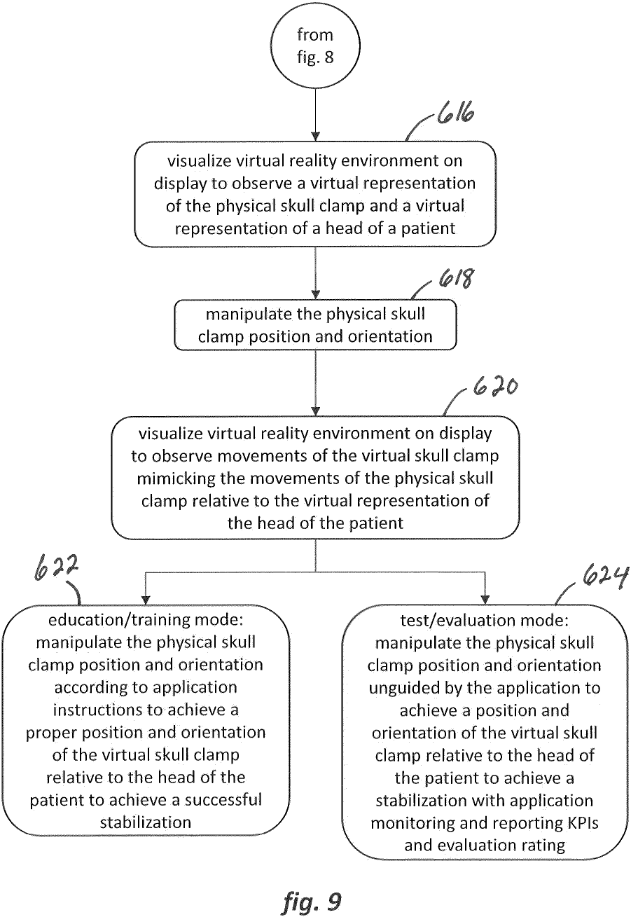

FIGS. 8-10 illustrate an exemplary use of the head stabilization simulation systems 10, 12 described herein. It should be understood that while the exemplary use or uses are shown and described herein in a sequence, this is for convenience of explanation and the precise order of the steps described herein can vary. Referring first to FIG. 8, a user desiring to conduct a head stabilization simulation, at step 600, launches application 500 from computing device 400. For an initial simulation, step 600 may include downloading and installing application 500 on computing device 400 in addition to executing application 500. For other repeat simulations, step 600 may simply include launching application 500 from computing device 400.

With application 500 launched, in some versions the user is presented with options to enter information about the stabilization simulation. In some versions application 500 includes predefined information from which the user can select. Such predefined information may be in the form of a menu of selections in a drop-down list or presented in some other easily selectable fashion. For example, step 602 involves selecting patient parameters. These may include, among others, things like the position of the patient (i.e., prone, supine, lateral recumbent, etc.), anatomical details of the patient (i.e., weight, age, size, bone density, etc.), and the surgical target locations (i.e., which area or region of the patient’s head or brain will be an operating site during the procedure). Step 604 involves selecting procedure parameters. These may include, among others, things like the procedure type and the planned path or trajectory to access the desired surgical sites. Step 606 involves selecting stabilization equipment parameters. These may include, among others, things like the skull clamp model being used, the adapter assembly configuration (i.e., single adapter assembly or dual adapter assembly), the stabilization features (i.e., pin configuration and/or pad configuration), and any other equipment that would be used in the actual stabilization (i.e., swivel adapter, base unit, table adapter, operating table, etc.). Step 608 involves selecting key process indicators (KPI) reporting. Based on the simulated stabilization conducted, this would include metrics like a surgeon ergonomic rating, a patient positioning rating, an equipment selection rating, a time, etc. Step 610 involves selecting test or evaluation parameters. This can be an optional step (along with any of steps 602, 604, 608) in preparation for later evaluating a user conducting a stabilization simulation. In some versions, step 610 involves determining a target or benchmark for each KPI. For instance, a target or benchmark for the surgeon ergonomic rating, the patient positioning rating, the equipment selection rating, and/or for the time it takes to complete the simulated stabilization can be determined and set in application 500. As discussed further below, with these target KPIs, application 500 can evaluate a user’s performance against these and output an overall score or rating for the user’s performance.

In terms of the KPIs discussed above, when considering the surgeon ergonomic rating, certain objective information can be used to assign or determine a rating and/or a target or benchmark. For example, in some instances it is desirable that the path or trajectory to be taken in the procedure is aligned with the direction of gravitational force. Where this is a higher degree of alignment between these two, the KPI rating would be assigned the more desirable value, which could be a higher or lower number depending on the approach. For instance in one version a rating of 100 may be consider the best (i.e., where 100 represents the least deviation between the path or trajectory and the direction of gravitational force), yet in some other versions a rating of 0 may be consider the best (i.e., where 0 represents the least deviation between the path or trajectory and the direction of gravitational force). Other information or factors that can be considered in the surgeon ergonomic KPI rating are the quantity and quality of the field of view. For instance, a better KPI rating would be assigned where the stabilization is achieved in a way that maximizes or meets a minimum acceptable level for the field of view (i.e., the stabilization equipment is not obstructing the surgeon’s view of the target surgical site). Furthermore, information such as the position and axis of the patient’s head may also impact the field of view and/or the overall surgeon ergonomic rating.

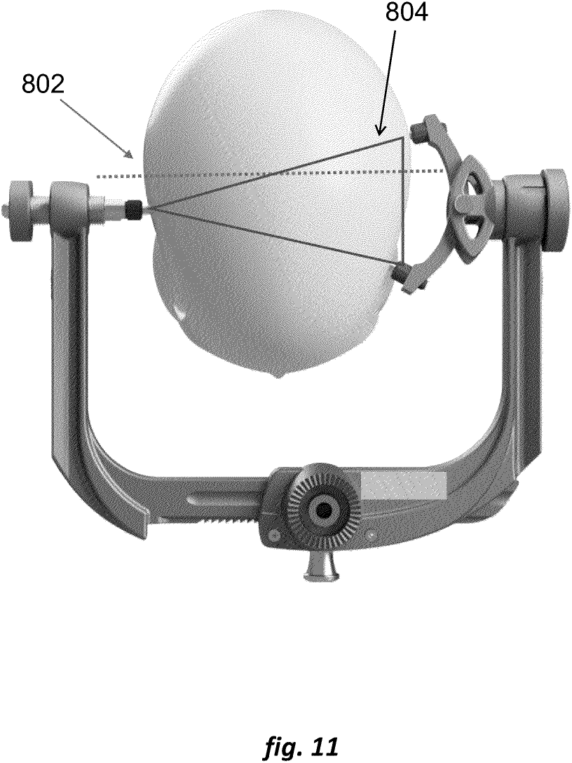

In terms of the KPI for the patient positioning rating, information about the ultimate position and orientation of the patient is used to assess a risk of injury or trauma to the patient. The time a patient is maintained in a given position may also be a factor. Where a simulated stabilization results in a low risk of injury to the patient, a more desirable KPI rating is assigned. Conversely, where there is a greater risk of injury or trauma to the patient, a less desirable KPI rating is assigned. In one example where the stabilization feature used comprises one or more skull pins, a pinning rating can be either a standalone KPI or incorporated as a factor within the KPI for the patient positioning rating. When considering a pinning rating, factors to consider can include, among others, the pinning geometry (i.e., is a triangular pinning geometry present), the pinning location (i.e., are two of three pins located below the equator of the skull; does the pinning location avoid the forehead area that would leave cosmetic scarring as opposed to being concealed within the hairline), and the pinning depth relative to the bone structure (i.e., would the insertion depth and required force be suitable for the patient’s given bone structure). Still other factors or considerations for pinning location include if the pins are located in the safe zone. A safe zone is understood to be a location that avoids vascular and neuronal structures in the head as well as areas where the bone is fragile such that a fracture or injury could occur when pinning the bone. The safe zone may also take into account avoiding prior craniotomy defects, subcutaneous artificial or foreign structures, and the orbit and the pinna. Still other factors or considerations for pinning location include if the pins of the 2-pin rocker arm stabilization assembly are placed first followed by the counter lateral single pin, if the single pin is placed in an area of ridged bone, if all pins avoid neurovascular structures as mentioned above, if the pins are placed in a same sided triangle, if two of three pins are placed below the equator, and/or if penetration angle of the pins is ninety degrees or perpendicular to the skull. FIG. 11 depicts an exemplary stabilization with markings illustrating a skull clamp and the equator 802 and triangular pinning geometry 804 as mentioned above.

In terms of the KPI for the equipment selection rating, application 500 can include a library or database of suggested or recommended equipment based on the specific patient and procedure input. Based on this, a more desirable KPI rating would be assigned when the user selects equipment that matches the suggested or recommended equipment. In view of the teachings herein, various other parameters that can be included with application 500 will be apparent to those of ordinary skill in the art.

With the various parameters discussed above entered within application 500, at step 612 computing device 400 is mounted to skull clamp 100, 200 via the respective adapter assembly 120, 220, 221. With computing device 400 connected with the adapter assembly, at step 614, the simulation begins. Additionally, as mentioned above, the order of the steps depicted in FIG. 8 is not fixed such that the steps may occur in any order and are not required to be carried out precisely sequentially as shown.

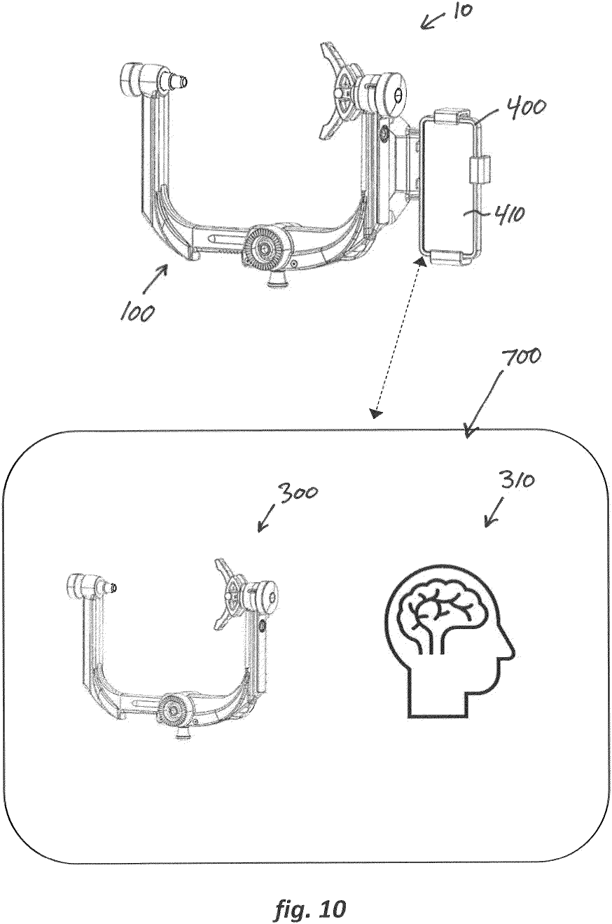

Referring to FIGS. 9 and 10, at step 616 the user visualizes a virtual reality environment 700 on a display that in one version is the display 410 of computing device 400. In other versions this display can be a display that is external to computing device 400, i.e., a glasses or goggles display, a remote monitor, etc. In the virtual reality environment 700 shown on the display, the user observes a virtual skull clamp 300 that represents the physical skull clamp 100, 200. Additionally, the user observes a virtual representation of the patient’s head 310 in virtual reality environment 700.

At step 618, the user manipulates the physical skull clamp 100, 200 to change its position and/or orientation. For instance, the user would grasp skull clamp 100 or skull clamp 200 depending on the setup, and move the skull clamp to change its position and/or to change its orientation. As mentioned above, a change in position refers to a change in the spatial three-dimensional coordinates while a change in orientation refers to a change in the tilt or angulation about axes in the three-dimensional system, i.e., X, Y, and Z axes defined e.g., by the skull clamp.

At step 620, the user visualizes virtual reality environment 700 on the display to observe movements of virtual skull clamp 300 relative to the virtual head of the patient 302 based on or mimicking the movements of physical skull clamp 100 or skull clamp 200 as the case may be.

At step 622, application 500 is in an education or training mode. In this mode, the user manipulates the position and/or orientation of skull clamp 100 or skull clamp 200, as the case may be depending on which skull clamp is being used. This manipulation is done according to instructions from application 500. In other words, in this education or training mode of using application 500, application 500 instructs or guides the user as to how to manipulate the position and/or orientation of skull clamp 100, 200 to achieve an acceptable virtual patient stabilization or an optimal virtual patient stabilization in virtual reality environment 700.

At step 624, application 500 is in a test or evaluation mode. In this mode, the user’s performance of conducting a virtual stabilization is assessed. In this mode application 500 does not instruct or guide the user in a step-by-step fashion as described above with respect to step 622. Instead, application 500 monitors the movements the user makes with skull clamp 100, 200 to achieve the virtual stabilization and thereafter can provide feedback as an output in terms of KPI reporting, etc. Similarly as mentioned above, the order of the steps depicted in FIG. 9 is not fixed such that the steps may occur in any order and are not required to be carried out precisely sequentially as shown.

In depicting virtual skull clamp 300 within virtual reality environment 700 based on movements of the physical skull clamp 100, 200, such correlation and depiction is achievable because computing device 400 is connectable with skull clamp 100, 200 at a known position and orientation relative to skull clamp 100, 200 as discussed above. Based on this baseline or known initial position and orientation correlation between computing device 400 and skull clamp 100, 200, and with computing device 400 mounted to skull clamp 100, 200, as computing device 400 with its various sensors described above is moved to alter its position and/or orientation, the same change in position and/or orientation is ascribed to skull clamp 100, 200. Furthermore, this data is an input to application 500 such that virtual skull clamp 300 is depicted such that it mimics or mirrors the movements of physical skull clamp 100, 200. As shown in exemplary virtual reality environment 700, virtual skull clamp 300 is shown without adapter assemblies shown to provide a more realistic visual experience since the actual skull clamps used in the actual procedures may not include the adapter assemblies. In view of the teachings herein, various other ways to correlate the position and orientation of a physical skull clamp to a virtual skull clamp and depict the same using application 500 and computing device 400 or a similar application and/or computing device will be apparent to those of ordinary skill in the art.

### V. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. A system for simulating stabilizing a head of a patient, the system comprising:
(a) a skull clamp; and
(b) an adapter assembly connectable with the skull clamp at a defined position and orientation relative to the skull clamp, wherein the adapter assembly is configured to selectively receive a computing device configured to access an application for correlating a first position and a first orientation of the computing device with a second position and a second orientation of the skull clamp.

2. The system of claim 1, wherein the application is configured to educate or train a user to use the skull clamp to stabilize the head of the patient.

3. The system of claim 2, wherein the application allows depiction of the skull clamp and the second position and the second orientation of the skull clamp within a virtual reality environment that shows the second position and the second orientation of the skull clamp relative to the head of the patient.

4. The system of claim 3, wherein the depiction of the skull clamp in the virtual reality environment shows the skull clamp without the adapter assembly being part of, or connected with, the skull clamp.

5. The system of claim 1, wherein the skull clamp comprises a first arm and a second arm that are adjustable relative to each other to adjust a distance between the first arm and the second arm.

6. The system of claim 1, wherein at least a portion of the adapter assembly is formed unitarily with the skull clamp.

7. The system of claim 1, wherein the skull clamp comprises two or more stabilization assemblies, wherein each of the two or more stabilization assemblies comprises a stabilizing feature configured to contact the head of the patient to stabilize the head of the patient.

8. The system of claim 1, wherein the adapter assembly defines a void space that allows the user to grasp a portion of the skull clamp directly without contacting the adapter assembly.

9. The system of claim 1, wherein the computing device includes a processor, a set of executable instructions, a memory configured to store the set of executable instructions, and a display, wherein the set of executable instructions provide access to the application.

10. The system of claim 1, further comprising:
(c) the computing device selectively connectable with the adapter assembly; and
(d) the application accessible from the computing device, wherein the application comprises a simulation software for simulating stabilizing the head of the patient using the skull clamp.

11. The system of claim 1, further comprising a display in communication with the computing device, wherein the display is configured to depict a virtual reality environment where the skull clamp and the head of the patient are represented virtually, and movement of the skull clamp correlates to movement of the skull clamp represented virtually in the virtual reality environment.

12. A system for simulating stabilizing a head of a patient, the system comprising:
(a) a skull clamp; and
(b) a pair of adapter assemblies connectable with the skull clamp at a respective first defined position and a first defined orientation relative to the skull clamp and a second defined position and a second defined orientation relative to the skull clamp, wherein a first adapter assembly of the pair of adapter assemblies is configured to selectively receive a first computing device configured to access an application for correlating a first position and a first orientation of the first computing device with a second position and a second orientation of the skull clamp, and wherein a second adapter assembly of the pair of adapter assemblies is configured to selectively receive a second computing device configured to communicate with the first computing device.

13. The system of claim 12, wherein the second computing device is configured to access the application for correlating a third position and a third orientation of the second computing device with the second position and the second orientation of the skull clamp.

14. The system of claim 13, wherein the application is configured to correlate the first position and the first orientation of the first computing device with the third position and the third orientation of the second computing device.

15. The system of claim 14, wherein the skull clamp comprises a first arm and a second arm, wherein the correlation of the first position and the first orientation of the first computing device with the third position and the third orientation of the second computing device indicates a distance between the first arm and the second arm of the skull clamp.

16. The system of claim 15, wherein the first arm and the second arm are adjustable relative to each other to adjust a distance between the first arm and the second arm.

17. The system of claim 12, wherein the application allows depiction of the skull clamp and the second position and the second orientation of the skull clamp within a virtual reality environment that shows the second position and the second orientation of the skull clamp relative to a depiction of the head of the patient.

18. The system of claim 12, wherein the skull clamp comprises two or more stabilization assemblies, wherein each of the two or more stabilization assemblies comprises a stabilizing feature configured to contact the head of the patient to stabilize the head of the patient.

19. The system of claim 15, wherein at least a portion of the first adapter assembly of the pair adapter assemblies is formed unitarily with the first arm, and at least a portion of the second adapter assembly of the pair of adapter assemblies is formed unitarily with the second arm.

20. The system of claim 12, wherein each of the adapter assemblies defines a void space that allows the user to grasp the skull clamp directly without contacting the adapter assemblies.

21. The system of claim 17, wherein the depiction of the skull clamp in the virtual reality environment shows the skull clamp without the adapter assemblies being part of, or connected with, the skull clamp.

22. The system of claim 12, wherein the skull clamp is a rapid prototype or a 3D printed skull clamp.

23. A method of simulating stabilizing a head of a patient using a skull clamp having an adapter assembly, the method comprising the steps of:
(a) launching an application from a computing device, wherein the application comprises a simulation software for stabilizing the head of the patient;
(b) mounting the computing device with the adapter assembly;
(c) visualizing a virtual reality environment on a display to observe a virtual skull clamp represented by the skull clamp and to observe a virtual head of a virtual patient;
(d) manipulating the skull clamp;
(e) visualizing the virtual reality environment on the display to observe movements of the virtual skull clamp mimicking movements of the skull clamp relative to the virtual head of the virtual patient; and
(f) manipulating the skull clamp according to application instructions to achieve a desired position and orientation of the virtual skull clamp relative to the virtual head of the virtual patient to achieve a virtual stabilization.

24. The method of claim 23, further comprising selecting stabilization equipment parameters within the application.

25. The method of claim 23, further comprising selecting patient parameters within the application.

26. The method of claim 23, further comprising selecting procedure parameters within the application.

27. The method of claim 23, further comprising selecting KPI reporting within the application.

28. The method of claim 23, further comprising selecting test or evaluation parameters within the application.
